(19) United States
(12) Patent Application Publication
(10) Pub. No.: US 2023/0181781 A1
(43) Pub. Date: Jun. 15, 2023

(54) Room Sanitizing Assembly

(71) Applicants: Salvador Guerra, Corona, CA (US); Raul G. Paredes, Anaheim, CA (US)

(72) Inventors: Salvador Guerra, Corona, CA (US); Raul G. Paredes, Anaheim, CA (US)

(21) Appl. No.: 17/547,368

(22) Filed: Dec. 10, 2021

(51) Int. Cl. A61L 2/16; A61L 2/24; A61L 2/26

(52) U.S. Cl. CPC A61L 2/16; A61L 2/24; A61L 2/26; A61L 2202/25; A61L 2202/14; A61L 2202/15

(57) ABSTRACT

A room sanitizing assembly includes a reservoir that contains a fluid disinfectant. A pump is coupled to the reservoir for pumping the fluid disinfectant outwardly from the reservoir. A pipe is fluidly coupled to the pump such that the pipe receives the fluid disinfectant from the pump. The pipe is routed to exit the dedicated room in the building having the pipe extending over a ceiling of a common room of the building. A plurality of nozzles is each fluidly coupled to the pipe such that each of the nozzles receives the fluid disinfectant. Each of the nozzles extends through the ceiling of the common room of the building such that the nozzles spray the fluid disinfectant into the common room to sanitize the common room thereby reducing transmission of infectious diseases.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to sanitizing device and more particularly pertains to a new sanitizing device for sanitizing a room in a building. The device includes a reservoir that contains a fluid disinfectant, a fluid pump for pumping the fluid disinfectant, a distribution pipe and a plurality of nozzles that are each fluidly coupled to the distribution pipe. The distribution pipe extends over a common room in the building and the nozzles extend through a ceiling of the common room for spraying the fluid disinfectant into the common room.

### (2) Description of Related Art Including Information Disclosed Under 37 CFR 1.97 and 1.98

The prior art relates to sanitizing devices including a variety of sanitizing devices that includes a pump, distribution pipes and spray nozzles. In each case the distribution pipes are routed through walls and ceilings of a building. The prior art discloses a variety of aircraft sanitizing devices that include a reservoir, a pump and spray nozzles, each being integrated into an aircraft, for automatically sanitizing an interior of the aircraft.

### (h) BRIEF SUMMARY OF THE INVENTION

An embodiment of the disclosure meets the needs presented above by generally comprising a reservoir that contains a fluid disinfectant. A pump is coupled to the reservoir for pumping the fluid disinfectant outwardly from the reservoir. A pipe is fluidly coupled to the pump such that the pipe receives the fluid disinfectant from the pump. The pipe is routed to exit the dedicated room in the building having the pipe extending over a ceiling of a common room of the building. A plurality of nozzles is each fluidly coupled to the pipe such that each of the nozzles receives the fluid disinfectant. Each of the nozzles extends through the ceiling of the common room of the building such that the nozzles spray the fluid disinfectant into the common room to sanitize the common room thereby reducing transmission of infectious diseases.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a room sanitizing assembly according to an embodiment of the disclosure.

FIG. 2 is a perspective in-use view of an embodiment of the disclosure.

FIG. 3 is a cross sectional view taken along line 3-3 of FIG. 2 of an embodiment of the disclosure.

FIG. 4 is a schematic view of an embodiment of the disclosure.

### (j) DETAILED DESCRIPTION OF THE INVENTION

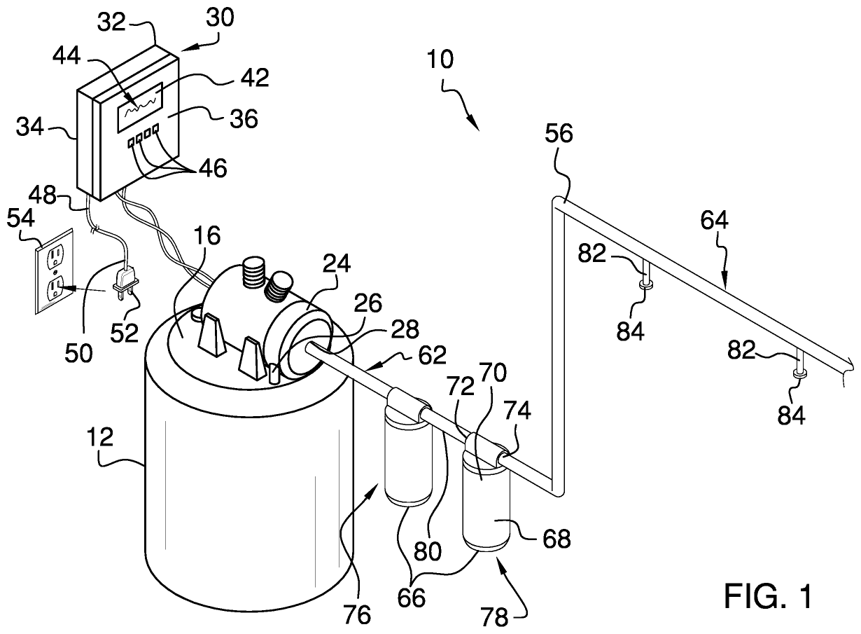

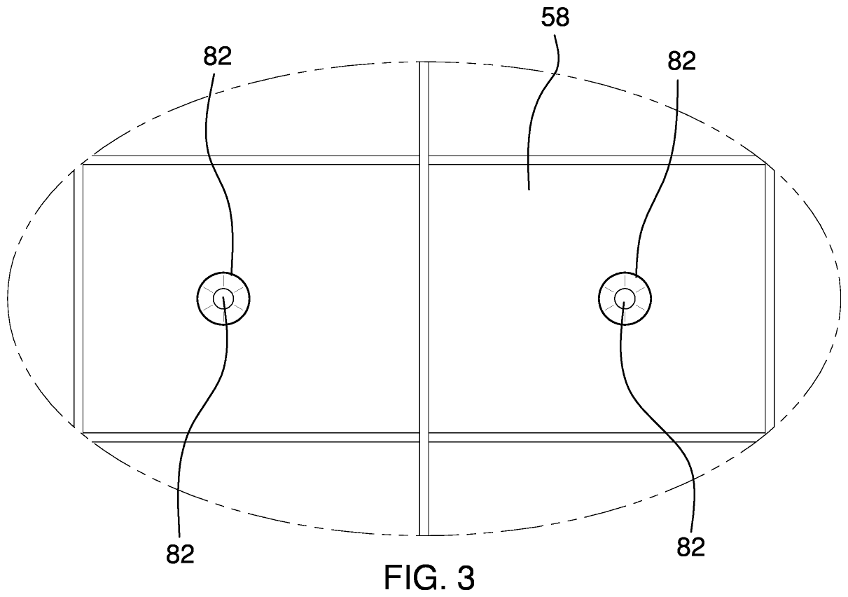

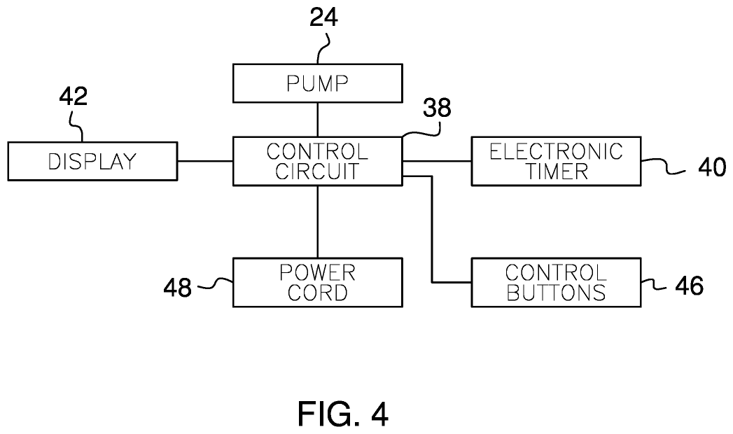

With reference now to the drawings, and in particular to FIGS. 1 through 4 thereof, a new sanitizing device embodying the principles and concepts of an embodiment of the disclosure and generally designated by the reference numeral 10 will be described.

As best illustrated in FIGS. 1 through 4, the room sanitizing assembly 10 generally comprises a reservoir 12 which contains a fluid disinfectant 14. The fluid disinfectant 14 may be a non-toxic sanitizer that kills bacteria and viruses but is non-toxic to human beings. The reservoir 12 has a top wall 16 and the reservoir 12 is positioned in a dedicated room 18 in a building 20 such that the reservoir 12 is concealed from observers. The top wall 16 of the reservoir 12 has a fill spout 22 extending into an interior of the reservoir 12 for filling the reservoir 12. A pump 24 is provided and the pump 24 is coupled to the reservoir 12. The pump 24 is in fluid communication with the interior of the reservoir 12 for pumping the fluid disinfectant 14 outwardly from the reservoir 12. The pump 24 is positioned on the top wall 16 of the reservoir 12 and the pump 24 has an inlet 26 and an outlet 28. Furthermore, the inlet 26 is in fluid communication with the interior of the reservoir 12. The pump 24 may comprise an electric fluid pump or the like.

A control unit 30 is positioned in the dedicated room 18 of the building 20 and the control unit 30 is in communication with the pump 24. The control unit 30 actuates the pump 24 at pre-determined times of day and the control unit 30 de-actuates the pump 24 after a pre-determined duration of time. The control unit 30 comprises a housing 32 that has a rear wall 34 and a front wall 36. The rear wall 34 is mounted to a vertical support surface 35 in the dedicated room 18 such that the front wall 36 is accessible to a user. A control circuit 38 is positioned in the housing 32 and the control circuit 38 is electrically coupled to the pump 24. The control circuit 38 receives an on input and the control circuit 38 receives an off input. The pump 24 is turned on when the control circuit 38 receives the on input and the pump 24 is turned off when the control circuit 38 receives the off input.

The control unit 30 includes an electronic timer 40 is positioned in the housing 32 and the electronic timer 40 is electrically coupled to the control circuit 38. The control circuit 38 receives the on input when the electronic timer 40 begins counting down a pump duration of time. Moreover, the control circuit 38 receives the off input when the electronic timer 40 completes counting down the pump duration of time. A display 42 is coupled to the front wall 36 of the housing 32 such that the display 42 is visible to the user. The display 42 is electrically coupled to the control circuit 38 and the display 42 displays indicia 44 comprising numbers and letters for communicating operational parameters of the pump 24 including, but not being limited to, the pump duration of time.

The control unit 30 includes a plurality of control buttons 46 that is each movably integrated into the front wall 36 of the housing 32. Each of the control buttons 46 is electrically coupled to the control circuit 38 and the control buttons 46 facilitates the pump duration of time to be selected. Moreover, the control buttons 46 facilitate a duration of time to be determined between sequential pump durations of time. In this way the pump 24 can be repeatedly actuated at selected times of the day. A power cord 48 is coupled to and extends away from the rear wall 34 of the housing 32 and the power cord 48 is electrically coupled to the control circuit 38. The power cord 48 has a distal end 50 with respect to the rear wall 34 and a male power plug 52 is electrically coupled to the distal end 50 for plugging into a power source 54 comprising a female electrical outlet.

A pipe 56 is fluidly coupled to the pump 24 such that the pipe 56 receives the fluid disinfectant 14 from the pump 24. The pipe 56 is routed to exit the dedicated room 18 in the building 20 having the pipe 56 extending over a ceiling 58 of a common room 60 of the building 20 such that the pipe 56 is concealed from occupants of the common room 60. The pipe 56 has a supply section 62 and a distribution section 64, and the supply section 62 is fluidly coupled to the outlet of the pump 24. The distribution section 64 extends over the ceiling 58 of the common room 60 of the building 20.

A pair of filters 66 is each fluidly coupled to the distribution pipe 56 such that the fluid disinfectant 14 is routed through the filters 66. Each of the filters 66 is comprised of a particle filtering material to filter particles from the fluid disinfectant 14 to inhibit the distribution pipe 56 from becoming clogged with the particles. Each of the filters 66 has a cylindrical housing 68 and the cylindrical housing 68 has an outer wall 70. Furthermore, each of the filters 66 has a filtration capability sufficient to remove the SARS-CoV-2 (COVID 19) virus from the distribution pipe 56. Additionally, the outer wall 70 of the cylindrical housing 68 has an entrance 72 extending into an interior of the cylindrical housing 68 and the outer wall 70 of the cylindrical housing 68 has an exit 74 extending into the interior of the cylindrical housing 68.

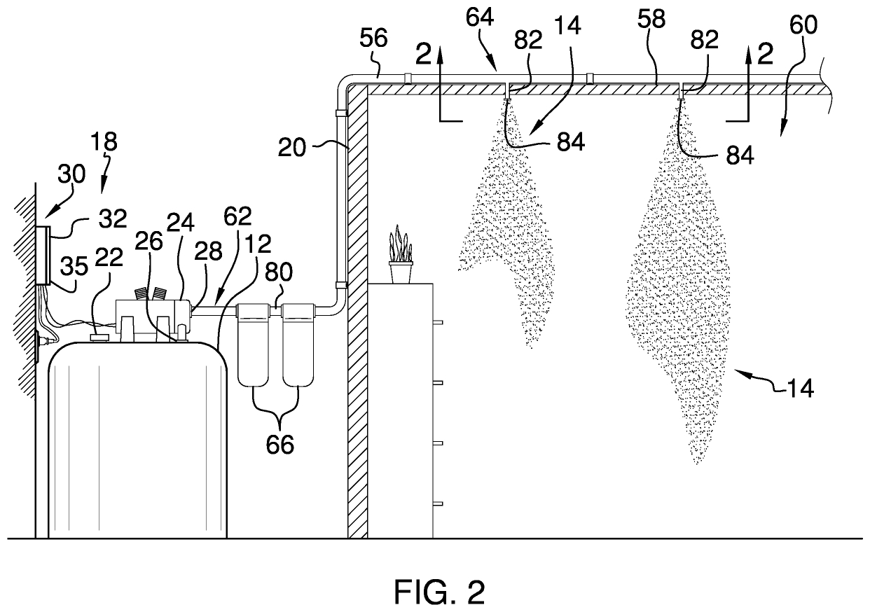

The pair of filters 66 includes a first filter 76 and a second filter 78, and the supply section 62 of the pipe 56 is fluidly coupled to the entrance 72 associated with the first filter 76. The exit 74 associated with the first filter 76 is in fluid communication with the entrance 72 associated with the second filter 78. As is most clearly shown in FIGS. 1 and 2, a short section of pipe 80 may extend between the exit 74 associated with the first filter 76 and the entrance 72 associated with the second filter 78. The distribution section 64 of the pipe 56 is fluidly coupled to the exit 74 associated with the second filter 78. Each of the filters 66 may comprise a fluid permeable membrane which can pass the fluid disinfectant 14 while capturing particles of greater than approximately 0.01 microns.

A plurality of nozzles 82 is provided and each of the nozzles 82 is fluidly coupled to the pipe 56 such that each of the nozzles 82 receives the fluid disinfectant 14. Each of the nozzles 82 extends through the ceiling 58 of the common room 60 of the building 20 such that the nozzles 82 spray the fluid disinfectant 14 into the common room 60. In this way the fluid disinfectant 14 can sanitize the common room 60 thereby reducing transmission of infectious diseases. Each of the nozzles 82 has a distal end 84 with respect to the pipe 56 and the distal end 84 of each of the nozzles 82 is open. The nozzles 82 are spaced apart from each other and are distributed over an entire length of the distribution section of the pipe 56. Furthermore, the distribution pipe 56 and the nozzles 82 may be strategically positioned to ensure horizontal surfaces in the common room 60, such as table tops or the like, are covered with the fluid disinfectant 14.

In use, the control unit 30 is programmed to establish the duration of time that the pump 24 should be actuated along with the number of times over the course of the day that the pump 24 is actuated. In this way the pump 24 automatically supplies the fluid disinfectant 14 to the plurality of nozzles 82 to facilitate the fluid disinfectant 14 to sanitize the common room 60. Thus, the health and safety of occupants of the common room 60 is enhanced with respect to being protected from infectious diseases. The reservoir 12 is filled with the fluid disinfectant 14 when the reservoir 12 becomes depleted.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A room sanitizing assembly for sanitizing a room with a fluid disinfectant, said assembly comprising:
a reservoir containing a fluid disinfectant;
a pump being coupled to said reservoir, said pump being in fluid communication with an interior of said reservoir for pumping said fluid disinfectant outwardly from said reservoir;
a control unit being positioned in the dedicated room of the building, said control unit being in communication with said pump, said control unit actuating said pump at pre-determined times of day, said control unit de-actuating said pump after a pre-determined duration of time;
a pipe being fluidly coupled to said pump such that said pipe receives said fluid disinfectant from said pump, said pipe being routed to exit the dedicated room in the building having said pipe extending over a ceiling of a common room of the building wherein said pipe is configured to be concealed from occupants of the common room;
a pair of filters, each of said filters being fluidly coupled to said distribution pipe such that said fluid disinfectant is routed through said filters, each of said filters being comprised of a particle filtering material to filter particles from said fluid disinfectant wherein said filters are configured to inhibit said distribution pipe from becoming clogged with the particles; and
a plurality of nozzles, each of said nozzles being fluidly coupled to said pipe such that each of said nozzles receives said fluid disinfectant, each of said nozzles extending through the ceiling of the common room of the building such that said nozzles spray said fluid disinfectant into the common room wherein said fluid disinfectant is configured to sanitize the common room thereby reducing transmission of infectious diseases.

2. The assembly according to claim 1, wherein:
said reservoir has a top wall, said reservoir being positioned in a dedicated room in a building wherein said reservoir is configured to be concealed from observers; and
said pump is positioned on said top wall of said reservoir, said pump having an inlet and an outlet, said inlet being in fluid communication with said interior of said reservoir.

3. The assembly according to claim 1, wherein said control unit comprises:
a housing having a rear wall and a front wall, said rear wall being mounted to a vertical support surface in the dedicated room wherein said front wall is configured to be accessible to a user;
a control circuit being positioned in said housing, said control circuit being electrically coupled to said pump, said control circuit receiving an on input, said control circuit receiving an off input, said pump being turned on when said control circuit receives said on input, said pump being turned off when said control circuit receives said off input;
an electronic timer being positioned in said housing, said electronic timer being electrically coupled to said control circuit, said control circuit receiving said on input when said electronic timer begins counting down a pump duration of time, said control circuit receiving said off input when said electronic timer completes counting down said pump duration of time;
a display being coupled to said front wall of said housing wherein said display is configured to be visible to the user, said display being electrically coupled to said control circuit, said display displaying indicia comprising numbers and letters for communicating operational parameters of said pump including said pump duration of time;
a plurality of control buttons, each of said control buttons being movably integrated into said front wall of said housing, each of said control buttons being electrically coupled to said control circuit, said control buttons facilitating said pump duration of time to be selected, said control buttons facilitating a duration of time between sequential pump durations of time to be selected; and
a power cord being coupled to and extending away from a back wall of said housing, said power cord being electrically coupled to said control circuit, said power cord having a distal end with respect to said back wall, said distal end having a male power plug being electrically coupled to said distal end wherein said male power plug is configured to be plugged into a power source comprising a female electrical outlet.

4. The assembly according to claim 2, wherein:
said pipe has a supply section and a distribution section, said supply section being fluidly coupled to said outlet of said pump, said distribution section extending over the ceiling of the common room of the building; and
each of said filters has a cylindrical housing, said cylindrical housing having an outer wall, said outer wall of said cylindrical housing having an entrance extending into an interior of said cylindrical housing, said outer wall of said cylindrical housing having an exit extending into said interior of said cylindrical housing, said pair of filters including a first filter and a second filter, said supply section of said pipe being fluidly coupled to said entrance associated with said first filter, said exit associated with said first filter being in fluid communication with said entrance associated with said second filter, said distribution section of said pipe being fluidly coupled to said exit associated with said second filter.

5. A room sanitizing assembly for sanitizing a room with a fluid disinfectant, said assembly comprising:
a reservoir containing a fluid disinfectant, said reservoir having a top wall, said reservoir being positioned in a dedicated room in a building wherein said reservoir is configured to be concealed from observers;
a pump being coupled to said reservoir, said pump being in fluid communication with an interior of said reservoir for pumping said fluid disinfectant outwardly from said reservoir, said pump being positioned on said top wall of said reservoir, said pump having an inlet and an outlet, said inlet being in fluid communication with said interior of said reservoir;
a control unit being positioned in the dedicated room of the building, said control unit being in communication with said pump, said control unit actuating said pump at pre-determined times of day, said control unit de-actuating said pump after a pre-determined duration of time, said control unit comprising:
a housing having a rear wall and a front wall, said rear wall being mounted to a vertical support surface in the dedicated room wherein said front wall is configured to be accessible to a user;
a control circuit being positioned in said housing, said control circuit being electrically coupled to said pump, said control circuit receiving an on input, said control circuit receiving an off input, said pump being turned on when said control circuit receives said on input, said pump being turned off when said control circuit receives said off input;
an electronic timer being positioned in said housing, said electronic timer being electrically coupled to said control circuit, said control circuit receiving said on input when said electronic timer begins counting down a pump duration of time, said control circuit receiving said off input when said electronic timer completes counting down said pump duration of time;
a display being coupled to said front wall of said housing wherein said display is configured to be visible to the user, said display being electrically coupled to said control circuit, said display displaying indicia comprising numbers and letters for communicating operational parameters of said pump including said pump duration of time;
a plurality of control buttons, each of said control buttons being movably integrated into said front wall of said housing, each of said control buttons being electrically coupled to said control circuit, said control buttons facilitating said pump duration of time to be selected, said control buttons facilitating a duration of time between sequential pump durations of time to be selected; and
a power cord being coupled to and extending away from a back wall of said housing, said power cord being electrically coupled to said control circuit, said power cord having a distal end with respect to said back wall, said distal end having a male power plug being electrically coupled to said distal end wherein said male power plug is configured to be plugged into a power source comprising a female electrical outlet;
a pipe being fluidly coupled to said pump such that said pipe receives said fluid disinfectant from said pump, said pipe being routed to exit the dedicated room in the building having said pipe extending over a ceiling of a common room of the building wherein said pipe is configured to be concealed from occupants of the common room, said pipe having a supply section and a distribution section, said supply section being fluidly coupled to said outlet of said pump, said distribution section extending over the ceiling of the common room of the building;
a pair of filters, each of said filters being fluidly coupled to said distribution pipe such that said fluid disinfectant is routed through said filters, each of said filters being comprised of a particle filtering material to filter particles from said fluid disinfectant wherein said filters are configured to inhibit said distribution pipe from becoming clogged with the particles, each of said filters having a cylindrical housing, said cylindrical housing having an outer wall, said outer wall of said cylindrical housing having an entrance extending into an interior of said cylindrical housing, said outer wall of said cylindrical housing having an exit extending into said interior of said cylindrical housing, said pair of filters including a first filter and a second filter, said supply section of said pipe being fluidly coupled to said entrance associated with said first filter, said exit associated with said first filter being in fluid communication with said entrance associated with said second filter, said distribution section of said pipe being fluidly coupled to said exit associated with said second filter; and
a plurality of nozzles, each of said nozzles being fluidly coupled to said pipe such that each of said nozzles receives said fluid disinfectant, each of said nozzles extending through the ceiling of the common room of the building such that said nozzles spray said fluid disinfectant into the common room wherein said fluid disinfectant is configured to sanitize the common room thereby reducing transmission of infectious diseases, each of said nozzles having a distal end with respect to said pipe, said distal end of each of said nozzles being open, said nozzles being spaced apart from each other and being distributed over an entire length of said distribution section of said pipe.

6. A room sanitizing system for sanitizing a room with a fluid disinfectant, said assembly comprising:
a building having a common room being configured to be occupied by people;
a reservoir containing a fluid disinfectant, said reservoir having a top wall, said reservoir being positioned in a dedicated room in said building wherein said reservoir is configured to be concealed from observers;
a pump being coupled to said reservoir, said pump being in fluid communication with an interior of said reservoir for pumping said fluid disinfectant outwardly from said reservoir, said pump being positioned on said top wall of said reservoir, said pump having an inlet and an outlet, said inlet being in fluid communication with said interior of said reservoir;
a control unit being positioned in the dedicated room of the building, said control unit being in communication with said pump, said control unit actuating said pump at pre-determined times of day, said control unit de-actuating said pump after a pre-determined duration of time, said control unit comprising:
a housing having a rear wall and a front wall, said rear wall being mounted to a vertical support surface in the dedicated room wherein said front wall is configured to be accessible to a user;
a control circuit being positioned in said housing, said control circuit being electrically coupled to said pump, said control circuit receiving an on input, said control circuit receiving an off input, said pump being turned on when said control circuit receives said on input, said pump being turned off when said control circuit receives said off input;
an electronic timer being positioned in said housing, said electronic timer being electrically coupled to said control circuit, said control circuit receiving said on input when said electronic timer begins counting down a pump duration of time, said control circuit receiving said off input when said electronic timer completes counting down said pump duration of time;
a display being coupled to said front wall of said housing wherein said display is configured to be visible to the user, said display being electrically coupled to said control circuit, said display displaying indicia comprising numbers and letters for communicating operational parameters of said pump including said pump duration of time;
a plurality of control buttons, each of said control buttons being movably integrated into said front wall of said housing, each of said control buttons being electrically coupled to said control circuit, said control buttons facilitating said pump duration of time to be selected, said control buttons facilitating a duration of time between sequential pump durations of time to be selected; and
a power cord being coupled to and extending away from a back wall of said housing, said power cord being electrically coupled to said control circuit, said power cord having a distal end with respect to said back wall, said distal end having a male power plug being electrically coupled to said distal end wherein said male power plug is configured to be plugged into a power source comprising a female electrical outlet;
a pipe being fluidly coupled to said pump such that said pipe receives said fluid disinfectant from said pump, said pipe being routed to exit said dedicated room in said building having said pipe extending over a ceiling of said common room of said building wherein said pipe is configured to be concealed from occupants of said common room, said pipe having a supply section and a distribution section, said supply section being fluidly coupled to said outlet of said pump, said distribution section extending over said ceiling of said common room of said building;
a pair of filters, each of said filters being fluidly coupled to said distribution pipe such that said fluid disinfectant is routed through said filters, each of said filters being comprised of a particle filtering material to filter particles from said fluid disinfectant wherein said filters are configured to inhibit said distribution pipe from becoming clogged with the particles, each of said filters having a cylindrical housing, said cylindrical housing having an outer wall, said outer wall of said cylindrical housing having an entrance extending into an interior of said cylindrical housing, said outer wall of said cylindrical housing having an exit extending into said interior of said cylindrical housing, said pair of filters including a first filter and a second filter, said supply section of said pipe being fluidly coupled to said entrance associated with said first filter, said exit associated with said first filter being in fluid communication with said entrance associated with said second filter, said distribution section of said pipe being fluidly coupled to said exit associated with said second filter; and
a plurality of nozzles, each of said nozzles being fluidly coupled to said pipe such that each of said nozzles receives said fluid disinfectant, each of said nozzles extending through said ceiling of said common room of said building such that said nozzles spray said fluid disinfectant into said common room wherein said fluid disinfectant is configured to sanitize said common room thereby reducing transmission of infectious diseases, each of said nozzles having a distal end with respect to said pipe, said distal end of each of said nozzles being open, said nozzles being spaced apart from each other and being distributed over an entire length of said distribution section of said pipe.
